(19) United States
(12) Plant Patent Application Publication
(10) Pub. No.: US 2003/0056270 P1
(43) Pub. Date: Mar. 20, 2003

(54) Chrysanthemum plant named 'Tutan Time'

(76) Inventor: Mark Roland Boeder, Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,489

(22) Filed: Sep. 19, 2001

(51) Int. Cl. A01H 5/00

(52) U.S. Cl. PLT/296

(57) ABSTRACT

A new variety of Chrysanthemum plant named ‘Tutan Time,’ having a medium upright and branching habit with a flat capitulum form of golden bronze single flowers with a green disc and darker centre.

### LATIN NAME OF THE GENUS AND SPECIES

Botanical classification: Dendranthema grandiflora.

### VARIETY DENOMINATION

The new Chrysanthemum variety denomination is ‘Tutan Time.’

### BACKGROUND OF THE INVENTION

The present invention comprises a new and distinct cultivar of Chrysanthemum botanically known as Dendranthema grandiflora, and referred to by the cultivar name ‘Tutan Time’.

‘Tutan Time’, identified as 20778-0 originated from a cross made by Mark Boeder, in a controlled breeding program in Chichester, West Sussex, United Kingdom. The female parent of the new variety is known as ‘Midas Time,’ and the male parent is the variety known as ‘Swing Time.’ The new variety ‘Tutan Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Tutan Time’ is a pot type of Chrysanthemum plant variety having an unusual flower form, giving a flat capitulum and golden bronze single type flowers.

### Comparison with Parent

Plants of the new Chrysanthemum variety ‘Tutan Time’ are similar to the parent cultivar ‘Midas Time’ in plant habitat and growth rate. In side-by-side comparisons in Chichester, West Sussex, United Kingdom, under commercial practice, plants of the new Chrysanthemum variety ‘Tutan Time’ differed from plants of the parent cultivar ‘Midas Time’ in the following characteristics.

1. The new variety ‘Tutan Time’ produces gold bronze single flowers whereas the parent cultivar ‘Midas Time’ produces gold single flowers with spoon shaped petals and green disc.

2. Plants of the new variety ‘Tutan Time’ have smaller and more inflorescence than plants of the parent cultivar ‘Midas Time.’

3. On average the new variety ‘Tutan Time’ has a two day faster response time compared to ‘Midas Time.’

### Comparison with Other Varieties

Plants of the new Chrysanthemum variety ‘Tutan Time’ are similar to the cultivar ‘Canton’ in plant habitat and growth rate. However, in side-by-side comparisons in Chichester, West Sussex, United Kingdom, under commercial practice, plants of the new Chrysanthemum variety ‘Tutan Time’ differed from plants of the cultivar ‘Canton’ in the following characteristics.

1. The new variety ‘Tutan Time’ produces gold bronze flowers, whereas the cultivar ‘Canton’ produces lavender white flowers.

2. Plants of the new variety ‘Tutan Time’ have smaller and shorter inflorescence than plants of the parent cultivar ‘Canton.’

3. The new variety ‘Tutan Time’ is more rounded in shape, with more even branching compared to the variety ‘Canton.’

### BRIEF DESCRIPTION OF ILLUSTRATIONS

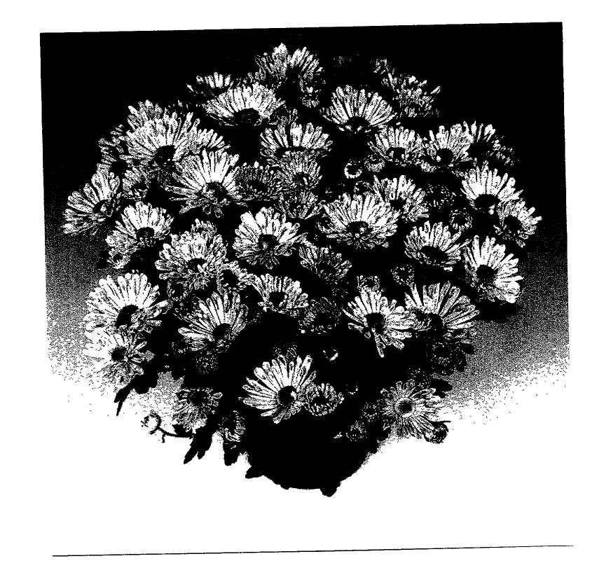

Typical specimens of the plant and flowers for the new Chrysanthemum variety ‘Tutan Time’ are shown in the accompanying photograph. The colours shown are as true as possible within the usual limits of this kind of illustration. FIG. 1 is a whole plant view of the new variety ‘Tutan Time’ grown in a pot. The plant shown in the illustration is 56 days from start of Short Days.

### DETAILED BOTANICAL DESCRIPTION

The following description of the new Chrysanthemum variety ‘Tutan Time’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as two B9 treatments at about 2000 ppm. To produce a commercial product the plants may be pinched once with the centre bud removed. The typical container size for commercial growth is 14 cm. It has been observed that the shelf life of the new variety is about 24 days with a response time of about 7½weeks. The new variety is suitable for growth in a temperature range of 16° C. to 25° C.

The new variety may be produced as a spray or disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot

Habit: Upright and branching

Height: Medium 18 to 21 cm.

Width: Near 25 cm.

Branching characteristics: Upright and spreading

Length of Lateral Branches: 10 to 15 cm.

Number of Breaks from Pinch: 5 to 7.

Stem Colour: 137 C.

Response Time: 51 days.

Vigour: Medium

Shelf Life: Near 22 days.

Disease: No observations

Pest: No Observations

Growth retardant type and treatment: B9 85% 2 applications of 2 gram/litre at 2 days, 21 days after propagating unrooted vegetative cuttings.

The plants were grown for two weeks in Long Day conditions (20 hours of light) and then transferred to Short Day conditions (13 hours of dark).

Propagation:

Type.—Vegetative propagation via stem cuttings

Time to rooting.—12-14 days with soil temperatures of 18° C.

Rooting habit.—Fine and fibrous

### Foliage

Number of Leaves per Lateral Branch: 7 to 11

Compound or single: Single

Arrangement of Leaves: Alternate

Shape of Leaf.—Typically 5 lobed, oblong ovate

Size of Leaf.—Width (cm): Near 4 to 5 cm Length (cm): 10 cm

Leaf Apex.—Acute

Base.—Obtuse

Attachment.—Petioled

Aspect.—Slightly undulating

Margin.—Palmately lobed

Surface characteristics.—Top: Pubescent Bottom: Pubescent

Petiole: Colour: Near 138 B Length: Near 2 cm

Venation: Net, prominent midvein under side.

Colour.—upper side: Near 138 B underside: Near 138 C

Colour: Mature Leaf, upper side: Near 138 A; under side: Near 138 B Young Leaf, upper side: Near 138 A; under side: Near 138 B

### Flower

Flower Appearance: Single Flower with quilled petals

Flower type: Single (Daisy)

Flower Form: Slightly cupped

Flower shape: Circular

Flower Habit: Upright

Number of blossoms per branch: 5 to 8

Inflorescence form: Cyme

Depth of fully expanded blossoms.—Near 1½cm

Diameter of fully expanded blossoms.—Near 7 cm

Sepals:

Number.—18 to 23

Colour.—Near 137 A

Length.—Near 1.2 cm

Width.—Near 0.3 cm

Texture/Appearance.—Pubescent

Peduncles: Peduncle length is shortest at the terminal flower and progressively longer moving down the stem; longest length is on average 10 cm; shortest length on average 5 cm.

Length.—6 cm

Colour.—Near 137 B

Surface.—Slightly pubescent

Habit.—Slightly curving

Strength.—Medium/Strong

Pedicel:

Length.—Terminal: Near 5 cm; Lateral: Near 7 cm

Colour.—Near 137 B

Surface.—Pubescent

Habit.—Slightly curving

Strength.—Medium/Strong

Ray Florets:

Form/Shape.—Straight, slightly spatulate

Texture/Appearance.—Matte

Number per flower.—30 to 45

Length.—1 to 2 cm; Width: 0.2 to 0.4 cm

Apex.—Rounded and Dentate

Base.—Tapered

Margin.—Entire

Disc florets:

Form/Shape.—Cylindrical

Texture/Appearance.—Shiny

Numberperflower.—25 to 30

Length.—Near 0.5 to 0.7 cm; Width: 0.05 to 0.1 cm

Diameter of disc.—Near 1.2 cm

Fragrance: Slight

Flower Bud (at onset of colour):

Length.—Near 0.7 cm

Diameter.—Near 1.0 cm

Form/shape.—Globular

General flower colour: Golden bronze

1. Ray florets, upper side: Immature: Near 25 A Mature: Near 23 A Older/Fading: Near 15 B

2. Ray florets, under side: Immature: Near 25 B Mature: Near 23 A Older/Fading: Near 15 B

3. Disc florets: Immature: Near 8 C Mature: Near 138 D Older/Fading: Near 8 C

4. Bud: Near 137 B Flower progression: Slight colour fading and flattening of flowers, with age

Reproductive Organs

Ray Florets.—Pistillate

Pistil number.—35 to 40

Stigma colour.—Near 6 C

Stigma shape.—Forked

Style Colour.—Near 6 C

Style Length.—Near 0.5 cm

Disc Florets: Pistillate

Pistil number.—<802

Stigma colour.—Near 6 C

Stigma shape.—Forked

Style Colour.—Near 6 C

Style Length.—Near 0.5 cm

Staminate.—

Stamen Number.—Approx. 150

Anther Shape.—Cylindrical

Anther Colour.—Near 9 A

Pollen Colour.—Bright yellow. Near 12 A

Pollen Quantity.—Little

What is claimed is:

1. A new and distinct variety of Chrysanthemum plant, substantially as described and illustrated herein.
